(19) United States
(12) Patent Application Publication
(10) Pub. No.: US 2014/0273007 A1
(43) Pub. Date: Sep. 18, 2014

(54) DEVICES AND METHODS FOR SCREENING NIPPLE ASPIRATE FOR MARKERS INDICATIVE OF RISK OF DEVELOPING BREAST CANCER

(71) Applicant: DR. SUSAN LOVE RESEARCH FOUNDATION, SANTA MONICA, CA (US)

(72) Inventor: Susan Love, Pacific Palisades, CA (US)

(73) Assignee: DR. SUSAN LOVE RESEARCH FOUNDATION, SANTA MONICA, CA (US)

(21) Appl. No.: 14/204,848

(22) Filed: Mar. 11, 2014

Related U.S. Application Data
(60) Provisional application No. 61/778,001, filed on Mar. 12, 2013.

(51) Int. Cl. G01N 33/574

(52) U.S. Cl. CPC G01N 33/57415
USPC 435/7.4; 422/69; 436/501; 435/287.2

(57) ABSTRACT

A diagnostic test system, apparatus and method for identifying individuals at risk for developing or having breast cancer by detection of at least one marker associated with increased breast cancer risk in nipple aspirate fluid is provided. A testing device for screening nipple aspirate fluid for the presence of biomarkers associated with breast cancer and methods of identifying individual biomarkers and biomarker panels for evaluating the risk that an individual has developed or will develop breast cancer by assaying nipple aspirate fluid is described.

### CROSS-REFERENCE TO RELATED APPLICATIONS

This application claims the priority benefit under 35 U.S.C. §119(e) of U.S. Provisional Application No. 61/778,001, filed Mar. 12, 2013, the entirety of which is hereby incorporated by reference herein. The disclosures of PCT Application No. PCT/US2008/063545, filed on May 13, 2008 and published as WO 2008/141318 on Nov. 20, 2008 and U.S. patent application Ser. No. 11/291,738, filed on Dec. 1, 2005 and issued as U.S. Pat. No. 7,487,779 on Feb. 10, 2009 is hereby incorporated herein in their entireties and made a part of the present application.

### BACKGROUND

1. Field

This application relates generally to devices, systems and methods for pre-screening individuals for markers associated with an increased risk for developing breast cancer, so that such individuals can be identified and referred for further evaluation.

2. Description of the Related Art

Breast cancer is a significant health problem in the industrialized western world, where it is the most common form of cancer among women in North America and almost all of Europe. It is estimated that each year the disease is diagnosed in over one million women worldwide and is the cause of death in over 400,000 women. The incidence of the disease is increasing in both industrialized and developing countries.

Although mammography screening has had some success in identifying tumors in asymptomatic women in the United States and Europe, it has limitations that make it less useful in the rest of the world. It is expensive to apply and requires trained technicians using high-tech equipment to achieve images that must then be interpreted by specialized radiologists. Mobile vans that take digital mammograms that can be read centrally have been developed in an attempt to overcome this problem, but these still require the use of scarce resources.

Further, mammography is most accurate in postmenopausal women, because their breasts tend to be less dense than those of women of reproductive age. In the United States and Europe, breast cancer is more common in postmenopausal Caucasian women, making mammographic screening a reasonable approach. For reasons not yet fully understood, in countries outside the United States and Europe, including many parts of the developing world, the majority of breast cancer cases occur in premenopausal women. In China, for example, the mean age of diagnosis is 48. For this population, mammography is not only expensive, but also not particularly sensitive.

Breast self-exam, whereby an individual massages the breast in search of tumors or other tissue abnormalities, might seem to be an answer to screening premenopausal women, since it is indeed cost-effective. Even clinical breast exam has limited evidence of effectiveness in reducing breast cancer mortality. Accordingly, a simple, inexpensive, accessible, and accurate test for breast cancer risk is needed.

### SUMMARY

Several embodiments relate to a biomarker-testing device for detecting in NAF, one or more biomarkers indicative of breast cancer or an increased risk of developing breast cancer.

Several embodiments relate to a biomarker-testing device comprising one or more test membranes for conducting one or more assays to detect in a NAF sample the presence of one or more biomarkers having a statistically significant correlation with an increased risk of having had developed or developing breast cancer. In some embodiments, the biomarker-testing device is configured to test 1, 2, 3, 4, 5, 6, 7, 8, 9, 10, 11, 12, 13, 14, 15, 16, 17, 18, 19, 20 or more biomarkers having a statistically significant correlation with an increased risk of having had developed or developing breast cancer. In some embodiments, the presence of specific biomarkers, specific combinations of biomarkers or specific subcombinations of biomarkers in NAF is indicative of a particular risk level for having had developed or developing breast cancer.

Several embodiments relate to a biomarker-testing device comprising one or more chambers for receiving 1, 2, 3, 4, 5, 6, 7, 8, 9, 10, or more biomarker testing membranes. In some embodiments, the biomarker-testing device is configured to accommodate substitution of testing membranes. In some embodiments, the biomarker-testing device is configured to accommodate substitution or inclusion of test membranes comprising assays to detect the presence of newly identified biomarkers having a statistically significant correlation with an increased risk of having had developed or developing breast cancer. In some embodiments, the biomarker-testing device is configured to accommodate substitution or inclusion of test membranes comprising assays to detect the presence of NAF components having an unknown breast cancer risk predictive value.

In one embodiment, the biomarker-testing device is designed to identify all individuals that test positive for at least one biomarker having a statistically significant correlation with an increased risk of having had developed or developing breast cancer. In another embodiment, the biomarker-testing device is designed to identify individuals that test positive for two or more, three or more, four or more, five or more, six or more, seven or more, eight or more biomarkers. In another embodiment, the biomarker-testing device is designed to identify individuals that test positive for all tested biomarkers.

Several embodiments relate to a single use biomarker-testing device comprising one or more test membranes. Several embodiments relate to a biomarker-testing device that is configured to reversibly receive one or more test membranes for conducting one or more assays to detect the presence of biomarkers having a statistically significant correlation with an increased risk of having had developed or developing breast cancer.

Several embodiments relate to a biomarker-testing device that enables simultaneous testing of multiple biomarkers in a format conducive to a home testing environment. In some embodiments, the biomarker-testing device enables simultaneous testing of two or more biomarkers. In some embodiments, the biomarker-testing device enables simultaneous testing of three or more biomarkers. In some embodiments, the biomarker-testing device enables simultaneous testing of four or more biomarkers. In some embodiments, the biomarker-testing device enables simultaneous testing of five or more biomarkers. In some embodiments, the biomarker-testing device enables simultaneous testing of six or more biomarkers. In some embodiments, the biomarker-testing device enables simultaneous testing of seven or more biomarkers. In some embodiments, the biomarker-testing device enables simultaneous testing of eight or more biomarkers. In some embodiments, the biomarker-testing device enables simultaneous testing of nine or more biomarkers. In some embodiments, the biomarker-testing device enables simultaneous testing of ten or more biomarkers.

In some embodiments, the biomarker-testing device is configured to detect the presence of one or more biomarkers selected from the group consisting of: 15-F2t-isoprostane; 2,6-Cyclolycopene-1,5-diol; 8-Epimer of prostaglandin F₂ₐ (8-iso-PGF(2α)); 8-Isoprostane; Albumin; alpha1-Acid glycoprotein (AAG); alpha1-Antichymotrypsin; alpha1-Antichymotrypsin; alpha1-Antitrypsin; alpha1B-Glycoprotein; alpha2-Glycoprotein(Zn), alpha2-HS-glycoprotein; alpha-Casein; alpha-Hemoglobin; alpha-Lactalbumin; alpha-L-fucosidase; Aluminum; Aminopeptidase N; Antigen p97-melanotransferrin; Apolipoprotein A-I; Apolipoprotein D; Apolipoprotein E; Basic fibroblast growth factor (bFGF); beta-2-Microglobulin; beta-Casein; Beta-globin; Beta-hemoglobin; Butyrophilin; Calgizzarin (S100 A11; MLN70); Calgranulin B (S100 A9); Cancer-associated serine protease-protecting peptide (CRISPP peptide); Carcinoembryonic antigen (CEA); Cathepsin D; CD14; CD34 (prominin); CD36; Ceruloplasmin (ferroxidase); Cholesterol; Cholesterol epoxides; Clusterin; Coagulation factor II; Collagen α1; Complement C3; Complement C4; Complement C7; Complement factor B; Complement factor D; C-reactive protein (CRP); Cyclooxygenase-2 (COX2); Dehydroepiandrosterone sulphate (DHEAS); Enhancer protein; Ephrin; Epidermal growth factor (EGF); Epidermal growth factor receptor 2 (HER-2/neu); Erythropoietin; Estradiol (E2); Estrone (E1); Ferritin; Fibrinogen gamma-B chain; Gelsolin; GOS reactivity; Gross cystic disease fluid protein 15 (GCDFP-15); Group IIa secretory phospholipase A2 (sPLA2-IIa); Haptoglobin; Hemopexin; Human glandular kallikrein 2 (hK2); Human glandular kallikrein 10 (hK10); Human glandular kallikrein 2 (hK2); Human glandular kallikrein 6 (hK6); Ig alpha chain (heavy); Ig gamma chain (heavy); Ig J chain; Ig kappa chain (light); Ig lambda chain (light); Ig mu chain (heavy); Immunoglobin (Ig); Immunoglobulin A (IgA); Immunoglobulin G (IgG); Immunoglobulin M (IgM); Insulin-like growth factor binding protein-3 (IGFBP-3); Interleukin-1 (IL-1); Interleukin-25 (IL-25); Interleukin-6 (IL-6); Iroquois-class homeodomain protein; kappa-Casein; Keratin 1; Keratin 2a; Lactate dehydrogenase enzymes (LDH); Lactoferrin; Lactose; Lactotransferrin; Leucine-rich α-2-glycoprotein; Lipophilin B; Macrophage inhibitory cytokine 1 (MIC-1); Mitogen-activated protein kinase (MAPK); Nuclear factor kappa B (NFκB); Osteopontin; p16; P-cadherin; Plasminogen activator inhibitor (PAI-1); Polymeric-immunoglobulin receptor; Progesterone; Prolactin; Prolactin-induced protein (GCDFP-15); Prostasin (serine protease 8); Prostate specific antigen (human glandular kallikrein 3) (PSA hK3); Prostate-specific antigen (PSA); Protein carbonyls; pS2; Pyruvate kinase isozyme M2; Retinoic acid receptor responder; S100 A11 (calgizzarin;MLN70); Superoxide dismutase-1; Testosterone; Thomsen-Freidenreich antigen (TF); Thymosin beta-4; Tn antigen (Tn); Transcobalamin I; Transferrin; Transforming growth factor-alpha (TGFα); Tumor necrosis factor alpha (TNFα); Tumor necrosis factor receptor (TNFR); Tumor-associated antigen 90K (Mac2 BP); Urokinase-type plasminogen activator (uPA); Urokinase-type plasminogen activator receptor (uPAR); Vascular endothelial growth factor (VEGF); Vitamin D-binding protein precursor; and Zinc alpha2-glycoprotein.

In some embodiments, the biomarker-testing device is configured to detect the presence of one or more biomarkers selected from the group consisting of: transferring, ferritin, erythropoietin, carcinoembryonic antigen (CEA), P-cadherin, Her2/neu, insulin-like growth factor binding protein-3 (IGFBP-3), p16, macrophage inhibitory cytokine 1 (MIC-1), cathepsin D, and esterase group IIa secretory phospholipase A2 (sPLA2-IIa.-). In some embodiments, the biomarker-testing device is configured to detect the presence of one or more biomarkers selected from the group consisting of: interleukin-1(IL-1), interleukin-6 (IL-6), interleukin-25 (IL-25), tumor necrosis factor alpha (TNFα), nuclear factor nuclear factor kappa-light-chain-enhancer of activated B cells (NFKB), cluster of differentiation 36 (CD36) and cyclooxygenase-2 (COX-2). In some embodiments, the biomarker-testing device is configured to detect the presence of one or more biomarkers selected from the group consisting of: EGF, CRP, and bFGF.

In some embodiments, the biomarker-testing device is configured to detect the presence of EGF, CRP, bFGF and one or more biomarkers selected from the group consisting of: 15-F2t-isoprostane; 2,6-Cyclolycopene-1,5-diol; 8-Epimer of prostaglandin F₂ₐ (8-iso-PGF(2α)); 8-Isoprostane; Albumin; alpha1-Acid glycoprotein (AAG); alpha1-Antichymotrypsin; alpha1-Antichymotrypsin; alpha1-Antitrypsin; alpha1B-Glycoprotein; alpha2-Glycoprotein(Zn), alpha2-HS-glycoprotein; alpha-Casein; alpha-Hemoglobin; alpha-Lactalbumin; alpha-L-fucosidase; Aluminum; Aminopeptidase N; Antigen p97-melanotransferrin; Apolipoprotein A-I; Apolipoprotein D; Apolipoprotein E; Basic fibroblast growth factor (bFGF); beta-2-Microglobulin; beta-Casein; Beta-globin; Beta-hemoglobin; Butyrophilin; Calgizzarin (S100 A11; MLN70); Calgranulin B (S100 A9); Cancer-associated serine protease-protecting peptide (CRISPP peptide); Carcinoembryonic antigen (CEA); Cathepsin D; CD14; CD34 (prominin); CD36; Ceruloplasmin (ferroxidase); Cholesterol; Cholesterol epoxides; Clusterin; Coagulation factor II; Collagen α1; Complement C3; Complement C4; Complement C7; Complement factor B; Complement factor D; C-reactive protein (CRP); Cyclooxygenase-2 (COX2); Dehydroepiandrosterone sulphate (DHEAS); Enhancer protein; Ephrin; Epidermal growth factor (EGF); Epidermal growth factor receptor 2 (HER-2/neu); Erythropoietin; Estradiol (E2); Estrone (E1); Ferritin; Fibrinogen gamma-B chain; Gelsolin; GOS reactivity; Gross cystic disease fluid protein 15 (GCDFP-15); Group IIa secretory phospholipase A2 (sPLA2-IIa); Haptoglobin; Hemopexin; Human glandular kallikrein 2 (hK2); Human glandular kallikrein 10 (hK10); Human glandular kallikrein 2 (hK2); Human glandular kallikrein 6 (hK6); Ig alpha chain (heavy); Ig gamma chain (heavy); Ig J chain; Ig kappa chain (light); Ig lambda chain (light); Ig mu chain (heavy); Immunoglobin (Ig); Immunoglobulin A (IgA); Immunoglobulin G (IgG); Immunoglobulin M (IgM); Insulin-like growth factor binding protein-3 (IGFBP-3); Interleukin-1 (IL-1); Interleukin-25 (IL-25); Interleukin-6 (IL-6); Iroquois-class homeodomain protein; kappa-Casein; Keratin 1; Keratin 2a; Lactate dehydrogenase enzymes (LDH); Lactoferrin; Lactose; Lactotransferrin; Leucine-rich α-2-glycoprotein; Lipophilin B; Macrophage inhibitory cytokine 1 (MIC-1); Mitogen-activated protein kinase (MAPK); Nuclear factor kappa B (NFκB); Osteopontin; p16; P-cadherin; Plasminogen activator inhibitor (PAI-1); Polymeric-immunoglobulin receptor; Progesterone; Prolactin; Prolactin-induced protein (GCDFP-15); Prostasin (serine protease 8); Prostate specific antigen (human glandular kallikrein 3) (PSA hK3); Prostate-specific antigen (PSA); Protein carbonyls; pS2; Pyruvate kinase isozyme M2; Retinoic acid receptor responder; S100 A11 (calgizzarin;MLN70); Superoxide dismutase-1; Testosterone; Thomsen-Freidenreich antigen (TF); Thymosin beta-4; Tn antigen (Tn); Transcobalamin I; Transferrin; Transforming growth factor-alpha (TGFα); Tumor necrosis factor alpha (TNFα); Tumor necrosis factor receptor (TNFR); Tumor-associated antigen 90K (Mac2 BP); Urokinase-type plasminogen activator (uPA); Urokinase-type plasminogen activator receptor (uPAR); Vascular endothelial growth factor (VEGF); Vitamin D-binding protein precursor; and Zinc alpha2-glycoprotein.

Several embodiments relate to a method of using biomarkers present in NAF for evaluating the risk that an individual has or will develop breast cancer. Several embodiments relate to a method for identifying members of a population at risk of developing breast cancer, and methods of calculating such risks, and advising individuals of such risks, based one the presence of one or more biomarkers or one or more panels of biomarkers in NAF.

Some embodiments relate to the identification of biomarkers and novel panels of biomarkers which can be used to evaluate the risk that an individual will develop breast cancer.

Several embodiments relate to a kit comprising a biomarker-testing device and instructions for use. Several embodiments relate to a kit comprising a nipple cap, a biomarker-testing device, and a buffer. In some embodiments, the kit further comprises instructions so that a woman can perform the test and view the results herself. In some embodiments, the kit further comprises a questionnaire relating to the subject's age, race, weight, family history, medical history, diet and/or habits. In some embodiments, the kit further comprises an envelope or other packaging for sending the biomarker-testing device to a medical professional following use. In some embodiments, the kit further comprises a postage-paid post card for reporting testing results to a medical professional. In some embodiments, the kit further comprises instructions for reporting testing results by email or for logging into a secure website.

### BRIEF DESCRIPTION OF THE DRAWINGS

Aspects of the disclosure will be readily apparent from the description below and the appended drawings, which are meant to illustrate and not to limit the disclosure, and in which:

FIG. 1 shows an illustration of one embodiment of a biomarker testing device comprising a well containing an aliquot of buffer, a well for receiving a nipple aspirate fluid collection membrane and test membranes for visualizing marker assays.

FIG. 2 shows an illustration of one embodiment of a biomarker testing device comprising a well for receiving a nipple aspirate fluid collection membrane and test membranes for visualizing marker assays.

FIG. 3A shows a diagrammatic illustration of a test membrane comprising a mobile gold-conjugated antibody capable of binding epitopes presented by molecules in the test sample and control line.

FIG. 3B shows a diagrammatic illustration of a test sample which does not contain target biomarker applied to a test membrane configured as shown in FIG. 3A.

FIG. 3C shows a diagrammatic illustration of a test sample containing target biomarker applied to a test membrane configured as shown in FIG. 3A.

### DETAILED DESCRIPTION

### Definitions

The following terms may be used in the present disclosure.

As used herein, “a” or “an” may mean one or more than one.

The term “antibody” as used herein shall be given its ordinary meaning and includes and refers to, without limitation, an immunoglobulin, whether natural or synthetically produced. The term also covers any polypeptide or protein derived from natural sources or synthetically produced having a binding domain, which is, or is homologous to, an antibody binding domain. Monoclonal, polyclonal and mixtures of monoclonal and polyclonal antibodies may be used. In some embodiments, fragments of antibody molecules may be used as specific binding reagents, including half antibody molecules and Fab, Fab′ or F(ab′)₂ fragments known in the art. In some embodiments, antibodies may be specifically reactive with various analytes whose detection is desired. Antibodies may be polyclonal or monoclonal and may be commercially available or may be obtained by mouse ascites, tissue culture or other techniques known to the art. In some embodiments, antibodies may be labeled with colloidal particles, fluorescent molecules, biotin and/or horse radish peroxidase.

The term “breast cancer” as used herein shall be given its ordinary meaning and includes and refers to, without limitation, any cancerous or malignant growth that begins in the breast including but not limited to non-invasive and metastatic breast cancers, ductal carcinoma in situ, lobular carcinoma in situ, invasive and/or infiltrating lobular and/or ductal carcinomas, inflammatory breast cancer, and medullary carcinoma. The term also includes breast cancers characterized as luminal subtype, basal A-like subtype, ER+, PgR+, ER−, PgR−, PTEN-, Her2/neu amplified, and/or erbB2 amplified. Breast cancer as used herein also includes different stages of breast cancer including but not limited to stage I, II (A and B), III (A, B and C) and IV.

The term “biomarker” as used herein shall be given its ordinary meaning and includes and refers to, without limitation, any substance or characteristic, which may be objectively measured and used as an indicator of a biological state, such as a disease state, normal biologic process, pathogenic process or pharmacologic response to therapeutic intervention. In some instances, biomarkers serve as prognostic indicators which can indicate the likelihood of developing a disease. Examples of biomarkers include, but are not limited to nucleic acids, such as genomic DNA cDNA or RNA and fragments thereof, proteins, polypeptides, carbohydrates, lipids, antibodies, hormones, metabolic products and combinations thereof. In some embodiments, the presence of a genetic mutation may serve as a biomarker indicating an increased risk of developing breast cancer. By way of a non-limiting example, mutations in the BRCA2 gene indicate an increased risk of developing breast cancer. In some embodiments, the presence of an RNA transcript, protein, polypeptide, carbohydrate, lipid, antibody, hormone, or metabolic product may serve as a biomarker indicating an increased risk of developing breast cancer. In some embodiments, the presence of a particular combination of genetic mutations, RNA transcripts, proteins, polypeptides, carbohydrates, lipids, antibodies, hormones, and metabolic products may serve as a biomarker indicating an increased risk of developing breast cancer. In some embodiments, a detected level of an RNA transcript, protein, polypeptide, carbohydrate, lipid, antibody, hormone, or metabolic product that falls above or below a threshold level may serve as a biomarker an increased risk of developing breast cancer. In some embodiments, a biomarker may be a genetic polymorphism.

The terms “biomarker assay” or “biomarker detection assay” as used herein shall be given its ordinary meaning and includes and refers to, without limitation, to any test that can identify a target biomarker.

The term “capture reagent” as used herein shall be given its ordinary meaning and includes and refers to, without limitation, any molecule or composition capable of recognizing a particular biomarker. In some embodiments, the capture reagent is an immunoglobulin which binds to an epitope of a biomarker. Other examples of capture reagents include receptors, antibodies, antigens, enzymes, Fab fragments, lectins, complementary nucleic acids, avidin, proteins, peptides and the like. In some embodiments, the capture reagent is immobilized on a membrane.

The term “epitope” as used herein shall be given its ordinary meaning and includes and refers to, without limitation, at least a portion of a molecule to which an antibody binds. Epitopes can be composed of sugars, lipids or amino acids.

The term “indicator reagent” as used herein shall be given its ordinary meaning and includes and refers to, without limitation, any molecule or combination of molecules capable of indicating the presence of one or more biomarkers. In some embodiments, an indicator reagent binds to a target biomarker and is conjugated, either directly or indirectly, to a label that is capable of producing a detectable signal. In some embodiments, an indicator reagent catalyzes a reaction that produces a detectable signal in the presence of a biomarker. In other embodiments, the biomarker catalyzes a reaction in the presence of an indicator reagent to produce a detectable signal. In preferred embodiments, the signal is visually detected. Examples of indicator reagents include, but are not limited to, labeled antigens, labeled antibodies, labeled proteins or protein fragments, labeled nucleic acids, dyes, enzymes, and enzymatic substrates. In some embodiments, the indicator reagent may comprise a first molecule that binds to the biomarker and a labeled second molecule that binds to the first molecule. In some embodiments, the first molecule may comprise an epitope tag, such as a Myc-tag, HA-tag, FLAG-tag, GST-tag, or 6× His tag. In some embodiments, the indicator reagent may comprise an antibody labeled with colloidal gold that binds to a target biomarker.

A “label” as used herein shall be given its ordinary meaning and includes and refers to, without limitation, any molecule that can produce a detectable signal. In some embodiments, colloidal gold particles are used as the label. Examples of suitable labels may include, but are not limited to, colloidal metal particles, minute colored particles, fluorescent molecules and colorimetric enzymes.

The terms nipple aspirate fluid (NAF) and ductal fluid are used interchangeably herein, and shall be given their ordinary meaning and include and refer to, without limitation, fluid obtained from breast ducts. NAF may be obtained by massaging the breast, aspiration, suction, rinsing or any other means known to those skilled in the art.

The term “nucleic acid” or “nucleic acid molecule,” as used herein shall be given its ordinary meaning and includes and refers to, without limitation, polynucleotides, such as deoxyribonucleic acid (DNA), complementary DNA (cDNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Nucleic acids can be either single stranded or double stranded.

The terms “patient” and “subject” may be used interchangeably and refer to a biological system from which a biological sample can be collected or to which a therapeutic agent can be administered. As used herein, a patient or subject can be human or a non-human mammal. Patients can include those that are healthy and those having a disease, such as breast cancer. Patients having a disease can include patients that have been diagnosed with the disease, patients that exhibit a set of symptoms associated with the disease, and patients that are progressing towards or are at risk of developing the disease.

A “polypeptide” as used herein shall be given its ordinary meaning and includes and refers to, without limitation, a polymer of amino acid residues joined by peptide bonds, whether produced naturally or synthetically.

A “protein” as used herein shall be given its ordinary meaning and includes and refers to, without limitation, a macromolecule comprising one or more polypeptide chains. A protein may also comprise non-peptide components, such as carbohydrate groups. Carbohydrates and other non-peptide substituents may be added to a protein by the cell in which the protein is produced, and will vary with the type of cell.

The term “confidence interval” (CI) as used herein shall be given its ordinary meaning and includes and refers to, without limitation, a random interval, derived from sample values, with a given probability of including the true population parameter. CIs for sensitivities and specificities can be determined from calculations using a normal approximation p±1.96(p(1−p)/n)^{1/2} where p is sensitivity/specificity and n is diseased/nondiseased sample size.

The term “false positive” (FP) as used herein shall be given its ordinary meaning and includes and refers to, without limitation, classifying a normal subject incorrectly as having or being at increased risk of developing breast cancer.

The term “false negative” (FN) as used herein shall be given its ordinary meaning and includes and refers to, without limitation, classifying a subject having breast cancer, or in some instances as having increased risk of developing breast cancer, incorrectly as not possessing indicators of breast cancer or having normal risk.

The term “true positive” (TP) as used herein shall be given its ordinary meaning and includes and refers to, without limitation, classifying a subject having breast cancer or having increased risk of developing breast cancer, correctly as possessing indicators of breast cancer.

The term “true negative” (TN) as used herein shall be given its ordinary meaning and includes or refers to, without limitation, classifying a normal subject correctly as not possessing indicators of breast cancer.

The term “increased risk” as used herein shall be given its ordinary meaning and includes, without limitation, an increased probability that an event, breast cancer, will occur relative to a control population. In some instances, the control population may be a general population of women. In some instances, the control population may consist of individuals having a particular age range, geographic location, race, ethnicity, medical history, family history, diet, or other unifying characteristic.

The term “sensitivity” as used herein shall be given its ordinary meaning and refers to or includes, without limitation, the percentage of subjects who have breast cancer or will develop breast cancer who are identified by the assay(s) provided in the biomarker testing device as having an increased risk of having had or having had developed breast cancer. Sensitivity is calculated by TP/(TP+FN)(100) or the true positive percentage of disease subjects. The term “sensitivity cut-off” refers to the value or level that identifies a selected baseline % of subjects who have breast cancer or an increased breast cancer risk.

The term “specificity” as used herein shall be given its ordinary meaning and includes, without limitation, the percentage of subjects who do not have and will not develop breast cancer who are identified by the assay(s) provided in the biomarker testing device as negative for risk markers. Specificity is calculated by TN/(TN+FP)(100) or the true negative percentage of normal subjects. The term “specificity cut-off” refers to the value or level that identifies a selected baseline % of subjects who do not have breast cancer.

### Overview

The breast comprises ductal lobular units coalescing into lobes that emerge through the nipple in about 6-8 accessible openings. Fluid obtained from these ducts, referred to herein as nipple aspirate fluid (NAF) or ductal fluid, contains substances that can be used as biomarkers to assess breast cancer risk. Clinical studies show that 80-90% of premenopausal women are able to express ductal fluid, although the volume of fluid obtained may be low. Embodiments described herein relate to methods, assays and medical testing devices that are sufficiently sensitive to detect the presence of biomarkers indicative of an increased risk of having had developed or developing breast cancer in a small volume of NAF, such as the small volumes obtainable by breast massage. Since according to some embodiments, only a small volume of NAF is required, the need for invasive procedures, such as, for example, ductal lavage and needle aspiration, mechanical devices (e.g., suctioning devices) and the like, is reduced or eliminated for NAF biomarker testing.

In addition to being cost-effective, less invasive, and convenient, collection of NAF by manual breast massage may also be advantageous because it avoids or helps reduce damage or irritation to sensitive breast tissue. Thus, in addition to providing the user with a more comfortable system, NAF, and by extension its component biological markers, may be obtained in a more undisturbed and stable state.

The embodiments described herein relate to assays and biomarker-testing devices for screening NAF to identify a subject at risk for developing breast cancer, to detect or diagnose breast cancer in a subject, to determine prognosis of a subject having breast cancer, and/or to monitor the therapeutic response of a subject to a breast cancer treatment. The assays and biomarker-testing devices described herein may further be utilized to screen NAF obtained from women with a range of breast cancer risk levels to identify biomarkers or panels of biomarkers having high sensitivity and specificity in identifying individuals having breast cancer or who are at risk for developing breast cancer.

Several embodiments described herein relate to a testing device that allows users to conduct a simple, inexpensive, and accurate screening assay for increased risk of breast cancer. Biomarker testing devices as described herein can be used by a woman to test her own nipple aspirate fluid for the presence of biomarkers indicative of an increased risk of breast cancer in the comfort of her own home and without the assistance of medical personnel. The testing devices described herein provide results that are easy to “read” and interpret. An individual obtaining a “positive” test result would be alerted to their increased risk for having developed or developing breast cancer. Such individual could then be further screened for a malignancy by a medical professional and/or be considered for preventative measures.

Several embodiments described herein relate to a testing device that is capable of identifying breast cancer risk in a subject with a specificity and/or sensitivity of at least 70%, 75% 80%, 85%, 90%, 95%, 96%, 97%, 98% or 99% and a confidence interval of at least 90%, 95%, 96%, 97%, 98% or 99%. In some embodiments, the biomarker testing device may perform a single biomarker assay capable of identifying breast cancer risk in a subject with a specificity and/or sensitivity of at least 70%, 75% 80%, 85%, 90%, 95%, 96%, 97%, 98% or 99% and a confidence interval of at least 90%, 95%, 96%, 97%, 98% or 99%. In several of the embodiments described herein, the testing device accommodates multiple biomarker screening assays. In some embodiments, the testing device may be optimized for providing an assessment of breast cancer risk by providing a panel of biomarker screening assays. In some embodiments, each biomarker assay of a biomarker assay panel is capable of identifying breast cancer risk in a subject with a specificity and/or sensitivity of at least 70%, 75% 80%, 85%, 90%, 95%, 96%, 97%, 98% or 99% and a confidence interval of at least 90%, 95%, 96%, 97%, 98% or 99%. In some embodiments, one or more individual biomarker assays included in a panel is capable of identifying breast cancer risk in a subject with less sensitivity and/or specificity than the panel. In some embodiments, the testing device may be optimized for providing an assessment of breast cancer risk by substituting one or more biomarker assays in a panel with one or more different biomarker assays. A panel of biomarker screening assays may be provided on one or more test strips. In some embodiments, a single biomarker assay is provided on a single test strip. In some embodiments, 2, 3, 4, 5, 6, 7, 8, 9, 10 or more biomarker assays are provided on a single test strip. In some embodiments, a panel of biomarker screening assays is provided on a single test strip.

Several embodiments described herein relate to a testing device comprising a single biomarker assay that is capable of identifying breast cancer risk in a subject with a specificity and/or sensitivity of at least 70%, 75% 80%, 85%, 90%, 95%, 96%, 97%, 98% or 99% and a confidence interval of at least 90%, 95%, 96%, 97%, 98% or 99% and one or more biomarker assays whose sensitivity or specificity for identifying breast cancer risk in a subject is unknown or poorly defined. Several embodiments described herein relate to a testing device comprising a biomarker assay panel capable of identifying breast cancer risk in a subject with a specificity and/or sensitivity of at least 70%, 75% 80%, 85%, 90%, 95%, 96%, 97%, 98% or 99% and a confidence interval of at least 90%, 95%, 96%, 97%, 98% or 99% and one or more biomarker assays whose sensitivity or specificity for identifying breast cancer risk in a subject is unknown or poorly defined. In some embodiments, the testing device may be optimized for providing an assessment of breast cancer risk by including a biomarker assay that is empirically shown to improve the sensitivity and/or specificity of the testing device.

Several embodiments described herein relate to a method of testing nipple aspirate fluid for the presence of biomarkers indicative of an increased risk of breast cancer. In some embodiments, NAF is collected from a subject and a biomarker assay is conducted utilizing one or more testing devices as described herein. In some embodiments, a subject self-expresses NAF by manual breast massage and assays collected NAF for biomarkers indicative of an increased risk of breast cancer utilizing a testing device as described herein. In other embodiments, NAF collection and assay may be conducted by healthcare professionals.

Several embodiments described herein relate to a method of using the assays and biomarker-testing devices described herein to identify biomarkers or panels of biomarkers having high sensitivity and specificity in identifying individuals having breast cancer or who are at risk for developing breast cancer by screening NAF obtained from women with a range of breast cancer risk levels and correlating the presence of a target biomarker with a risk level. Some embodiments relate to a method of identifying a target biomarker having high sensitivity and specificity in identifying breast cancer risk by screening NAF obtained from women with a range of breast cancer risk levels for the presence or absence of a target biomarker, correlating the presence or absence of the a target biomarker with a particular risk level and selecting a biomarker where a determination of its presence or absence in NAF is capable of identifying breast cancer risk with a specificity and/or sensitivity of at least 70%, 75% 80%, 85%, 90%, 95%, 96%, 97%, 98% or 99% and a confidence interval of at least 90%, 95%, 96%, 97%, 98% or 99%. Some embodiments relate to a method of identifying a biomarker panel having high sensitivity and specificity in identifying breast cancer risk by screening NAF obtained from women with a range of breast cancer risk levels for the presence or absence of 2, 3, 4, 5, 6, 7, 8, 9, 10, 11, 12, 13, 14, 15, 16, 17, 19, 20 or more target biomarker, correlating the presence or absence of a particular target biomarker with the presence or absence of one or more other target biomarkers in NAF obtained from women of a particular risk level and selecting a combination of biomarkers that is capable of identifying breast cancer risk with a specificity and/or sensitivity of at least 70%, 75% 80%, 85%, 90%, 95%, 96%, 97%, 98% or 99% and a confidence interval of at least 90%, 95%, 96%, 97%, 98% or 99%.

Collection of NAF

For use with the present embodiments, NAF can be obtained from the breast ducts of a subject either on their own or with the assistance of a medical professional and in any manner known in the art. For example, sufficient amounts of NAF can be obtained by massaging the breast. One technique includes pressing inward on the breast, moving from the chest wall toward the nipple. This massage helps move fluid from the back of the duct toward the natural openings in nipple, where the ductal fluid is expressed. Although breast massage is likely the most cost-effective and convenient option for obtaining NAF, other methods for and apparatuses directed at obtaining ductal fluid can also be used. Methods for obtaining NAF include, but are not limited to, aspiration, suction, lavage and rinsing. Likewise, apparatuses for obtaining NAF can include apparatuses employing aspiration, suction, rinsing and other means known to one of ordinary skill in the art. In some embodiments, sterile fluid can be added to dilute collected ductal fluid. In some embodiments, the ductal fluid is concentrated. It will be understood that the described embodiments making use of ductal fluid samples could also make use of diluted or concentrated samples.

In some embodiments, expressed NAF may be adsorbed onto a collection membrane, filter or pad. In some embodiments a collection membrane can be dabbed onto the nipple after manual massage to collect expressed NAF. In some embodiments a collection membrane may be positioned to cover the nipple during manual massage. In some embodiments, the collection membrane may be provided with an adhesive to secure the collection membrane to the breast. In some embodiments, the nipple may be covered by a device, such as a nipple cap, comprising a well to capture ductal fluid expressed by manual massage. In some embodiments, the well may comprise a collection membrane.

Other means for collecting NAF may also be used. For example, NAF may be collected from a nipple with a vial, capillary tube, needle, aspirator, pipette, suction device, etc. In some embodiments, expressed NAF may be rinsed from the nipple onto a collection membrane or into a collection container.

The collection membrane, filter or pad may be comprised of one or more hydrophilic, low protein-binding materials. Many such materials are known in the art. In some embodiments, the collection membrane is comprised of polymacron. In some embodiments, the collection membrane may be comprised of glass microfiber, polyvinylidene fluoride (PVDF), mixed cellulose esters, polycarbonate, cellulose fibers, nitrocellulose, polyester or any combination thereof.

Biomarker-Testing Devices

Several embodiments relate to a biomarker-testing device for performing one or more assays to detect the presence of one or more biomarkers indicative of breast cancer risk in a NAF sample.

In several embodiments, the biomarker-testing device comprises a case comprising one or more compartments for receiving one or more test membranes for conducting a biomarker detection assay. Some embodiments relate to a biomarker-testing device comprising a case that is configured to reversibly receive one or more test membranes placed there by a manufacturer, researcher, healthcare professional, subject or other user. Some embodiments relate to a biomarker-testing device comprising a case configured for single use comprising one or more compartments comprising one or more test membranes for conducting one or more biomarker detection assays. In some embodiments, the case of the biomarker-testing device is configured such that all or a portion of a test membrane compartment is exposed. In some embodiments, the case of the biomarker-testing device is configured to fully enclose one or more test membrane compartments. In some embodiments, the case of the biomarker-testing device further comprises one or more transparent windows for viewing test membrane(s). Screens and associated processors, circuitry and power sources for detecting and displaying biomarker assay results are well known in the art and may be incorporated into the case of a biomarker-testing device as described herein. The case may be any shape, for example square, rectangular, triangular, circular, oval or amorphous, and may be composed of any suitable material or combination of materials.

In some embodiments, a test membrane compartment of the case is configured to receive a NAF sample. In some embodiments, the case comprises one or more wells for receiving a sample of NAF. In some embodiments, one or more wells of the biomarker-testing device are self-contained. In some embodiments, the case is configured such that a test membranes or a portion thereof is coupled to a sample receiving well. In some embodiments, the case comprises one or more channels coupling a sample receiving well to the test membrane(s). In some embodiments, upon addition of NAF and/or buffer to a well of the biomarker-testing device, the well(s) are fluidly coupled to the test membrane(s). In some embodiments, one or more wells of the biomarker-testing device may comprise buffer suitable for dilution and/or elution of NAF. In some embodiments, one or more wells of the biomarker-testing device may comprise an indicator reagent.

In some embodiments, the biomarker-testing device comprises a wicking member for obtaining a sample of NAF. In some embodiments, the case of the biomarker-testing device is configured such that a wicking member is fluidly coupled to the test membrane(s) upon being soaked with NAF and/or buffer. In some embodiments, the wicking member is exposed and extends from an end of the case.

In some embodiments, the biomarker-testing device and associated buffer can be stored at room temperature for long periods of time, for example, more than 6 weeks, more than 6 months, more than 12 months, more than 2 years, or longer, without diminishing the activity of the biomarker detection assay(s).

In an embodiment depicted at FIG. 1, the case of the biomarker-testing device comprises a first segment and a second segment, wherein the first segment comprises a self-contained well and the second segment comprises one or more test membranes coupled to a well for receiving a NAF sample, such as a NAF-containing membrane, wherein the first and second segment are connected by a flexible hinge and wherein the case or housing of the biomarker-testing device is configured such that a surface of the first segment can be positioned to oppose a surface of the second segment, bringing buffer contained in the well of the first segment in contact with the NAF sample contained in the well of the second segment. In some embodiments, the buffer containing well is provided with a removable lid or cap (not shown). In some embodiments, the well of the first segment is configured to reversibly connect to the well of the second segment.

In some embodiments, the biomarker testing device visually communicates test results by appearance of a color change on a biomarker testing membrane. In some embodiments, the biomarker testing device communicates test results by visual appearance of numerals, letters, dots, lines, patterns or symbols on a biomarker testing membrane. In some embodiments, the biomarker testing device indicates a “positive” result at least one tested biomarker is detected in the NAF sample. In some embodiments, the biomarker testing device indicates a “positive” result when two or more, three or more, four or more, or five or more tested biomarkers are detected in the NAF sample. In some embodiments, the biomarker testing device indicates a “positive” result when all tested biomarkers are detected in the NAF sample.

Biomarker Testing Membranes

The biomarker testing devices described herein comprise or may be reversibly provided with one or more test membranes for conducting one or more breast cancer biomarker detection assays. In some embodiments, a test membrane may be configured to detect a single biomarker associated with an increased risk of developing or having had developed breast cancer in a NAF sample. In some embodiments, a test membrane may be configured to detect a panel of biomarkers that when present together in a NAF sample is predictive of an increased risk of developing or having breast cancer. A test membrane may be configured to detect 1, 2, 3, 4, 5, 6, 7, 8, 9, 10, 11, 12, 13, 14, 15, 16, 17, 18, 19, 20, 21, 22, 23, 24, 25 or more biomarkers. In a preferred embodiment, a test membrane may be configured to perform one or more biomarker detection assays performed under compatible reaction conditions.

A test membrane may be configured to employ a specific binding interaction, a chemical reaction, an enzymatic reaction or any combination thereof to detect the presence of one or more target biomarkers in a NAF sample. A test membrane may be composed of any suitable material or combination of materials. In one embodiment, the test membrane is a nitrocellulose membrane. A test membrane may comprise one or more reagents, such as capture reagents, enzymes, substrates, dyes, and chemical reaction components, which specifically interact with a complementary target biomarker. It is contemplated that one or more test membranes can be designed to detect any number of target biomarkers, for which a specific binding partner or assay reagent exists or can be synthesized.

In some embodiments, a test membrane is configured to perform a biomarker detection assay by employing a specific binding interaction. A test membrane may comprise one or more capture reagents that specifically recognize and physically interact with a complementary target biomarker. The capture reagent binds, usually non-covalently, to a complementary target biomarker so as to form a complex that can be detected, either directly or indirectly. In a preferred embodiment, a target biomarker and capture reagent may participate in an immunological reaction, e.g. antigen-antibody. In other embodiments, a target biomarker and capture reagent may participate in a non-immunological reaction, e.g., avidin and biotin, cell surface receptor and an effector agent, binding of complementary nucleotides, protein-protein interaction and so forth. The members of the specific binding reaction (target biomarker and capture reagent) may include, for example, antigens and antibodies (realizing that the terms antigen and antibody are not mutually exclusive since antibodies can act as antigens for other antibodies) or fragments thereof, hormones and hormone receptors or fragments thereof, signaling molecules and their receptors or fragments thereof, complementary polynucleotides, lipids and lipid binding proteins, and sugars and lectins. The target biomarker and capture reagent may correspond to either member of the specific binding pair. For example, a hormone receptor molecule can act as a capture reagent in an assay for the corresponding hormone biomarker. Alternatively, a hormone could act as a capture reagent in an assay for the corresponding hormone receptor biomarker. The biomarker may be a peptide, protein, carbohydrate, glycoprotein, lipid, nucleic acid, steroid, or other organic or inorganic molecule for which a specific binding partner exists or can be synthesized.

In some embodiments, one or more capture reagents are immobilized directly or indirectly on a test membrane. The capture reagent may be immobilized on a test membrane by any method, for example, absorption, adsorption, evaporative deposition from a volatile solvent solution, immunological immobilization or covalent bonding to the test membrane. In some embodiments, one or more capture reagents are immobilized on a test membrane in a discrete capture site or “reaction zone” having an area substantially smaller than that of the entire surface area of the test membrane that facilitates the separation of the target biomarker from other components of the NAF sample. In some embodiments, the reaction zone may be formed by coating the upper surface of a defined region of the test membrane with a capture reagent or by entrapping a capture reagent within a matrix of the test membrane. When a NAP sample comprising a target biomarker is applied to the test membrane, the target biomarker becomes non-diffusively bound to capture reagent immobilized in the reaction zone, thus creating a high concentration of the target biomarker/capture reagent complex that can be detected with an indicator reagent.

In some embodiments, a test membrane is configured to perform a biomarker detection assay that relies on a non-binding interaction between a reagent and a target biomarker. A test membrane may comprise one or more assay reagents that specifically interact with a complementary target biomarker to produce a signal that may be detected either directly or with an indicator reagent. Examples of such reagents include, but are not limited to, enzymes, substrates, dyes, and chemical reaction components. In some embodiments, a test membrane may comprise a substrate that is cleaved or otherwise modified by a target enzyme biomarker. In some embodiments, a test membrane may comprise an enzyme that cleaves or otherwise modifies a target substrate biomarker. In some embodiments, a test membrane may comprise one or more chemical reaction components that specifically interact with a target biomarker to generate a reaction product. In some embodiments, the enzymatic or chemical reaction produces a signal that can be directly detected or detected with an indicator reagent. In some embodiments, the enzymatic or chemical reaction destroys a signal, for example, an epitope, binding member, carbohydrate or dye, which would otherwise be directly detectable or detectable with an indicator reagent. One or more assay reagents may be immobilized directly or indirectly on a test membrane by any method. For example, an assay reagent may be immobilized by absorption, adsorption, evaporative deposition from a volatile solvent solution, immunological immobilization or covalent bonding to a surface or within a test membrane. In some embodiments, one or more assay reagents may be immobilized in a discrete reaction zone having an area substantially smaller than that of the entire surface area of the test membrane.

In some embodiments, a reaction zone may comprise a single type of immobilized capture reagent or assay reagent that interact with a complementary target biomarker. In other embodiments, a reaction zone may comprise two or more different immobilized capture reagents and/or assay reagents that interact with one or more complementary target biomarkers. In some embodiments, 1, 2, 3, 4, 5, 6, 7, 8, 9, 10 or more capture reagents and/or assay reagents are immobilized in a single reaction zone. In some embodiments, a combination of capture reagents and/or assay reagents which interact with the same target biomarker may be used in a single reaction zone. In some embodiments, a combination of capture reagents and/or assay reagents which interact with different target biomarkers may be used in a single reaction zone. A reaction zone may be configured in any convenient pattern. For example, one or more capture reagents, assay reagents or any combination thereof may be distributed so that the reaction zone is configured as numerals, letters, dots, lines, patterns and symbols, which display a detectable signal upon completion of the assay. In some embodiments, the reaction zone is in the form of a single or double line.

In some embodiments, one or more capture reagents and/or assay reagents may be immobilized in different regions of the same test membrane to form multiple “reaction zones” such that multiple biomarker detection assays may be performed simultaneously. A test membrane may comprise 1, 2, 3, 4, 5, 6, 7, 8, 9, 10, 12, 13, 14, 15, 16, 17, 18, 19, 20, 21, 22, 23, 24, 25 or more reaction zones. A target biomarker may correspond to a single reaction zone or multiple reactions zones.

In some embodiments, a biomarker detection assay employs an indicator reagent comprising a label attached directly or indirectly to a molecule capable of binding to or otherwise interacting with a biomarker/capture reagent complex or a product of biomarker/assay reagent interaction. A detectable signal signifying the presence of the targeted biomarker in a NAF sample is produced where the indicator reagent is concentrated, for example, at a reaction zone. In some embodiments, an indicator reagent catalyzes a reaction producing a detectable signal in the presence of the biomarker or a product of a biomarker detection assay. Examples of indicator reagents include, but are not limited to, dyes, enzymes, substrates, labeled antigens, labeled antibodies directed to a biomarker or other antigen of interest, labeled antibody fragments, labeled proteins or protein fragments, and labeled nucleic acids. Examples of labels include, but are not limited to: enzymes; fluorescent, phosphorescent, and chemiluminescent dyes; latex and magnetic particles; dye crystalites, gold, silver, and selenium colloidal particles; metal chelates; coenzymes; electroactive groups; oligonucleotides, and fluorescent, phosphorescent, and chemiluminescent particles. In some embodiments, the indicator reagent is an antibody labeled with colloidal gold that binds to a target biomarker or biomarker/capture reagent complex. In some embodiments, the indicator reagent binds to the target biomarker at a site which does not interfere with the specific binding interaction between the target biomarker and the capture reagent. The indicator reagent may be provided, for example, on a collection membrane, in the NAF dilution/elution buffer, in a separate indicator solution, in a NAF-receiving well, or on the test membrane.

In several embodiments, a test membrane may further comprises one or more zones comprising a control molecule that either directly or indirectly produces a detectable signal in the presence of NAF regardless of biomarker composition. The control zone may be configured as numerals, letters, dots, lines, patterns and symbols, which display a detectable signal upon completion of the assay. In some embodiments, the control zone is in the form of a single line.

In some embodiments, an antibody directed to human immunoglobulin G (IgG), IgM, IgE, or IgA is immobilized in a control zone. Thus, when a NAF-containing sample contacts the test membrane, any immunoglobulin contained in the sample can be detected to determine whether the reagents are stable and the test is performing properly. In some embodiments, signal development, such as color development, at the control zone may be utilized to determine whether a sufficient amount of NAF was provided to the test membrane.

In some embodiments, a control sample of a targeted biomarker is immobilized in a control zone. Thus, an indicator reagent will bind or otherwise interact with the control sample of the biomarker regardless of whether or not a target biomarker happens to be present in the NAF sample. In this regard, signal development, such as color development, at the reaction zone may be compared with signal development at the control zone to determine whether the reagents are stable.

Several embodiments relate to a biomarker-testing device employing a “sandwich” immunoassay where the target biomarker is bound to an antibody as a capture agent and a labeled antibody as an indicator reagent.

In an embodiment depicted at FIG. 3A, a test membrane comprises a gold-conjugated indicator reagent (Antibody 1) that is capable of binding to both a target biomarker and the control molecule, a reaction zone comprising an immobilized capture reagent (Antibody 2) that is capable of binding the targeted biomarker and a control zone comprising an immobilized control molecule (Antibody 3) capable of binding to the indicator reagent. When, as depicted in FIG. 3B, a NAF sample that does not comprise a targeted biomarker is applied to the test membrane, the indicator reagent (Antibody 1) binds to the control molecule (Antibody 3), to produce a detectable signal in the control zone or “control line.” When, as depicted in FIG. 3C, a NAF sample comprising a targeted biomarker is applied to the test membrane, the indicator reagent (Antibody 1) is concentrated in the reaction zone through binding to the biomarker/capture reagent (Antibody 2) complex to produce a detectable signal “biomarker line” and in the control zone by binding to the control molecule (Antibody 3) to produce a detectable signal or “control line.”

It will be appreciated by those skilled in the art that while the embodiment depicted at FIG. 3 employs the formation of an immune complex to assay the biomarker composition of NAF, the assays and biomarker testing devices described herein are not restricted to these molecules. The present disclosure contemplates any assay in which a first member recognizes and specifically interacts with a second member. The first member can be conveniently termed a target biomarker and the second member a capture reagent or assay reagent. While several embodiments relate to an antigen and antibody as target biomarker and capture reagent, serving respective or alternative roles, the assays and biomarker-testing devices described herein can be used with a variety of capture reagents, assay reagents and biomarker molecules.

Biomarkers Indicating Increased Risk of Breast Cancer

One or more biomarker assays may be selected for screening individuals for breast cancer risk and inclusion in a biomarker-testing device as described herein based on factors such as reproducibility and ease of testing as well as the strength of a biomarker's association, either alone or in combination with other biomarkers, with a risk of developing or having developed breast cancer. In some instances, presence of a single biomarker is sufficiently indicative of a present and/or future disease state. In other instances, a specific combination or subcombination of biomarkers acts as an indicator of present and/or future disease state. The relevance or predictive impact of one biomarker might be altered based on the presence of one or more other biomarkers.

Biomarker assays may be selected for inclusion in a biomarker-testing device as described herein based on the “positive predictive value” (PPV) of the biomarker or a particular combination of biomarkers for having or developing breast cancer. The PPV of the presence of a biomarker or a particular combination of biomarkers in a NAF sample is based on the sensitivity (percentage of all subjects possessing the tested biomarker or combination of biomarkers that have or will develop breast cancer in a relevant time period (true positives)) and specificity (percentage of all subjects who do not have the tested biomarker or combination of biomarkers that will not develop breast cancer within a relevant time period (true negatives)). Presence in a NAF sample of a biomarker or a combination of biomarkers having a high PPV for having or developing breast cancer is indicative of increased breast cancer risk and the advisability of further screening and more frequent monitoring by a medical professional.

In some embodiments, biomarker assays may be selected for inclusion in a biomarker-testing device as described herein based on the “negative predictive value” (NPV) of the biomarker or a particular combination of biomarkers for having or developing breast cancer. The NPV of the presence of a biomarker or a particular combination of biomarkers in a NAF sample is based on the percentage of all subjects possessing the tested biomarker or combination of biomarkers that will not develop breast cancer within a relevant time period (sensitivity) and percentage of all subjects who do not have the tested biomarker or combination of biomarkers that have or will develop breast cancer in a relevant time period (specificity). Presence in a NAF sample of a biomarker or a combination of biomarkers having a high NPV for having or developing breast cancer is indicative of normal breast cancer risk.

While the predictive value of a biomarker or a particular combination of biomarkers for breast cancer risk depends both on the sensitivity and specificity of the test, sensitivity and specificity have a qualitatively inverse relationship. In some embodiments, sensitivity is favored over specificity for selection of biomarkers or particular combinations or subcombinations of biomarkers for use in NAF screening with a biomarker-testing device as described herein. In other embodiments, specificity is favored over sensitivity for selection of biomarker assays.

In some embodiments, the specificity cut-off level for selection of biomarker assays indicative of breast cancer for inclusion in a biomarker-testing device as described herein is greater than: 70%, 75% 80%, 85%, 90%, 95%, 96%, 97%, 98% or 99%. In some instances, a single biomarker is capable of identifying breast cancer with a specificity of at least 70%, 75% 80%, 85%, 90%, 95%, 96%, 97%, 98% or 99%, whereas, in other instances, a combination or plurality of biomarkers is used to obtain a specificity of at least 70%, 75% 80%, 85%, 90%, 95%, 96%, 97%, 98% or 99%. The biomarkers and combinations of biomarkers thus can be used to qualify breast cancer risk in a subject.

In some embodiments, the sensitivity cut-off level for selection of biomarker assays indicative of breast cancer for inclusion in a biomarker-testing device as described herein is greater than: 70%, 75% 80%, 85%, 90%, 95%, 96%, 97%, 98% or 99%. In some instances, a single biomarker is capable of identifying breast cancer with a sensitivity of at least 70%, 75% 80%, 85%, 90%, 95%, 96%, 97%, 98% or 99%, whereas, in other instances, a combination or plurality of biomarkers is used to obtain a sensitivity of at least 70%, 75% 80%, 85%, 90%, 95%, 96%, 97%, 98% or 99%.

In one embodiment, the biomarker assays selected for inclusion in a biomarker-testing device as described herein show a preference for providing a higher false positive rate, as opposed to a higher false negative rate. In one embodiment where a biomarker-testing device is designed to pre-screen individuals (or identify individuals for further screening) rather than to conduct a comprehensive screen or diagnosis, the test would tolerate a higher false positive rate than would be acceptable for a breast cancer diagnostic test (or other type of screening or diagnostic test) because it would be used as an initial screen to determine which individuals needed further evaluation. In some embodiments, the false positive rate is greater than 5%, 10%, 20% or 50%. In other embodiments, the biomarker assays selected for inclusion in a biomarker-testing device as described herein show a preference for providing a lower false positive rate. In some embodiments, the false positive rate is less than 10%, 9%, 8%, 7%,6%, 5%, 4%, 3%, 2%, or 1%.

A number of substances have been identified as being present at increased levels in NAF of women with increased risk of having had developed or developing breast cancer, such as those who have ductal carcinoma in situ, genetic risk factors, or who have had cancer in the contralateral breast. These substances include: the iron transport proteins, transferrin and ferritin; the erythropoiesis regulator, erythropoietin; the cell adhesion molecules carcinoembryonic antigen (CEA) and P-cadherin; the growth factor binding proteins Her2/neu and insulin-like growth factor binding protein-3 (IGFBP-3); the tumor suppressor gene p16; the cellular stress and immune response regulator macrophage inhibitory cytokine 1 (MIC-1); the lysosomal aspartic protease, cathepsin D; and the phospholipid esterase group IIa secretory phospholipase A2 (sPLA2-IIa). In addition, increasing evidence implicates inflammation as playing a role in setting the stage for breast cancer development, making markers of inflammation potential markers of increased risk of breast cancer. Markers of inflammation that when present in NAF might further serve as biomarkers indicative of breast cancer risk include: interleukin-1(IL-1), interleukin-6 (IL-6), interleukin-25 (IL-25), tumor necrosis factor alpha (TNFα), nuclear factor nuclear factor kappa-light-chain-enhancer of activated B cells (NFKB), cluster of differentiation 36 (CD36) and cyclooxygenase-2 (COX-2). Such substances may be used in the assays and biomarker-testing devices described herein to identify women having an increased risk of having had developed or developing breast cancer.

Several embodiments relate to a biomarker testing device configured to perform one or more assays to detect in a sample of NAF the presence of one or more biomarkers selected from the group consisting of: transferring, ferritin, erythropoietin, carcinoembryonic antigen (CEA), P-cadherin, Her2/neu, insulin-like growth factor binding protein-3 (IGFBP-3), p16, macrophage inhibitory cytokine 1 (MIC-1), cathepsin D, and esterase group IIa secretory phospholipase A2 (sPLA2-IIa).

Several embodiments relate to a biomarker testing device configured to perform one or more assays to detect in a sample of NAF the presence of one or more biomarkers selected from the group consisting of: interleukin-1(IL-1), interleukin-6 (IL-6), interleukin-25 (IL-25), tumor necrosis factor alpha (TNFα), nuclear factor nuclear factor kappa-light-chain-enhancer of activated B cells (NFKB), cluster of differentiation 36 (CD36) and cyclooxygenase-2 (COX-2).

Several embodiments relate to a biomarker testing device configured to perform one or more assays to detect in a sample of NAF the presence of one or more biomarkers selected from the group consisting of: EGF, CRP, and bFGF.

Several embodiments relate to a biomarker testing device configured to perform one or more assays to detect in a sample of NAF the presence of one or more biomarkers selected from the group consisting of: transferring, ferritin, erythropoietin, carcinoembryonic antigen (CEA), P-cadherin, Her2/neu, insulin-like growth factor binding protein-3 (IGFBP-3), p16, macrophage inhibitory cytokine 1 (MIC-1), cathepsin D, and esterase group IIa secretory phospholipase A2 (sPLA2-IIa); and one or more biomarkers selected from the group consisting of: interleukin-1(IL-1), interleukin-6 (IL-6), interleukin-25 (IL-25), tumor necrosis factor alpha (TNFα), nuclear factor nuclear factor kappa-light-chain-enhancer of activated B cells (NFKB), cluster of differentiation 36 (CD36) and cyclooxygenase-2 (COX-2); and optionally one or more biomarkers selected from the group consisting of: EGF, CRP, and bFGF.

Several embodiments relate to a biomarker testing device configured to perform one or more assays to detect in a sample of NAF the presence of one or more biomarkers selected from the group consisting of: EGF, CRP, and bFGF; and one or more biomarkers selected from the group consisting of: transferring, ferritin, erythropoietin, carcinoembryonic antigen (CEA), P-cadherin, Her2/neu, insulin-like growth factor binding protein-3 (IGFBP-3), p16, macrophage inhibitory cytokine 1 (MIC-1), cathepsin D, and esterase group IIa secretory phospholipase A2 (sPLA2-IIa). Several embodiments relate to a biomarker testing device configured to perform one or more assays to detect in a sample of NAF the presence of one or more biomarkers selected from the group consisting of: EGF, CRP, and bFGF; and one or more biomarkers selected from the group consisting of: interleukin-1(IL-1), interleukin-6 (IL-6), interleukin-25 (IL-25), tumor necrosis factor alpha (TNFα), nuclear factor nuclear factor kappa-light-chain-enhancer of activated B cells (NFKB), cluster of differentiation 36 (CD36) and cyclooxygenase-2 (COX-2).

Several embodiments relate to a biomarker testing device configured to perform one or more assays to detect in a sample of NAF the presence of one or more biomarkers selected from the group consisting of: 15-F2t-isoprostane; 2,6-Cyclolycopene-1,5-diol; 8-Epimer of prostaglandin F₂ₐ (8-iso-PGF(2α)); 8-Isoprostane; Albumin; alpha1-Acid glycoprotein (AAG); alpha1-Antichymotrypsin; alpha1-Antichymotrypsin; alpha1-Antitrypsin; alpha1B-Glycoprotein; alpha2-Glycoprotein(Zn), alpha2-HS-glycoprotein; alpha-Casein; alpha-Hemoglobin; alpha-Lactalbumin; alpha-L-fucosidase; Aluminum; Aminopeptidase N; Antigen p97-melanotransferrin; Apolipoprotein A-I; Apolipoprotein D; Apolipoprotein E; Basic fibroblast growth factor (bFGF); beta-2-Microglobulin; beta-Casein; Beta-globin; Beta-hemoglobin; Butyrophilin; Calgizzarin (S100 A11; MLN70); Calgranulin B (S100 A9); Cancer-associated serine protease-protecting peptide (CRISPP peptide); Carcinoembryonic antigen (CEA); Cathepsin D; CD14; CD34 (prominin); CD36; Ceruloplasmin (ferroxidase); Cholesterol; Cholesterol epoxides; Clusterin; Coagulation factor II; Collagen α1; Complement C3; Complement C4; Complement C7; Complement factor B; Complement factor D; C-reactive protein (CRP); Cyclooxygenase-2 (COX2); Dehydroepiandrosterone sulphate (DHEAS); Enhancer protein; Ephrin; Epidermal growth factor (EGF); Epidermal growth factor receptor 2 (HER-2/neu); Erythropoietin; Estradiol (E2); Estrone (E1); Ferritin; Fibrinogen gamma-B chain; Gelsolin; GOS reactivity; Gross cystic disease fluid protein 15 (GCDFP-15); Group IIa secretory phospholipase A2 (sPLA2-IIa); Haptoglobin; Hemopexin; Human glandular kallikrein 2 (hK2); Human glandular kallikrein 10 (hK10); Human glandular kallikrein 2 (hK2); Human glandular kallikrein 6 (hK6); Ig alpha chain (heavy); Ig gamma chain (heavy); Ig J chain; Ig kappa chain (light); Ig lambda chain (light); Ig mu chain (heavy); Immunoglobin (Ig); Immunoglobulin A (IgA); Immunoglobulin G (IgG); Immunoglobulin M (IgM); Insulin-like growth factor binding protein-3 (IGFBP-3); Interleukin-1 (IL-1); Interleukin-25 (IL-25); Interleukin-6 (IL-6); Iroquois-class homeodomain protein; kappa-Casein; Keratin 1; Keratin 2a; Lactate dehydrogenase enzymes (LDH); Lactoferrin; Lactose; Lactotransferrin; Leucine-rich α-2-glycoprotein; Lipophilin B; Macrophage inhibitory cytokine 1 (MIC-1); Mitogen-activated protein kinase (MAPK); Nuclear factor kappa B (NFκB); Osteopontin; p16; P-cadherin; Plasminogen activator inhibitor (PAI-1); Polymeric-immunoglobulin receptor; Progesterone; Prolactin; Prolactin-induced protein (GCDFP-15); Prostasin (serine protease 8); Prostate specific antigen (human glandular kallikrein 3) (PSA hK3); Prostate-specific antigen (PSA); Protein carbonyls; pS2; Pyruvate kinase isozyme M2; Retinoic acid receptor responder; S100 A11 (calgizzarin;MLN70); Superoxide dismutase-1; Testosterone; Thomsen-Freidenreich antigen (TF); Thymosin beta-4; Tn antigen (Tn); Transcobalamin I; Transferrin; Transforming growth factor-alpha (TGFα); Tumor necrosis factor alpha (TNFα); Tumor necrosis factor receptor (TNFR); Tumor-associated antigen 90K (Mac2 BP); Urokinase-type plasminogen activator (uPA); Urokinase-type plasminogen activator receptor (uPAR); Vascular endothelial growth factor (VEGF); Vitamin D-binding protein precursor; and Zinc alpha2-glycoprotein.

Kits

Another embodiment relates to a kit for screening NAF to identify a subject at risk for developing breast cancer, to detect or diagnose breast cancer in a subject, to determine prognosis of a subject having breast cancer, and/or to monitor the therapeutic response of a subject to a breast cancer treatment.

In some embodiments, the kit comprises one or more biomarker testing devices as described herein and instructional material. In some embodiments, the kit comprises a biomarker testing device, a NAF collection membrane and instructional material. In some embodiments, the kit comprises a biomarker testing device, a container of dilution buffer and instructional material. In some embodiments, the kit comprises a biomarker testing device, a collection membrane, a nipple cap and instructional material. In some embodiments, the kit comprises a biomarker testing device, a nipple cap and instructional material. In some embodiments, the kit comprises a biomarker testing device, a collection membrane, a nipple cap, a container of dilution buffer and instructional material. In some embodiments, the kit comprises a biomarker testing device, a nipple cap, a container of dilution buffer and instructional material. Kits according to any of the aforementioned embodiments may further comprise one or more of pipettes, gloves, a survey (either paper or online) of general health information and family history, and an addressed envelop for returning used devices. In some of the aforementioned embodiments, one or more biomarker testing devices included in the kit is configured as a single use device comprising one or more biomarker test membranes as described herein. In some of the aforementioned embodiments, the biomarker testing device is configured to receive one or more biomarker test membranes provided therewith. In some of the aforementioned embodiments, the one or more test membranes are provided based on criteria such as race, ethnicity, age, geographic location, previous test results, medical history, family history, genetic profile or other criteria. In some of the aforementioned embodiments, the one or more test membranes are provided based on criteria such as the racial profile, ethnic profile, genetic profile, age distribution or other characteristic of the population in the geographic area. In some of the aforementioned embodiments, the kit further comprises instructions for ordering additional biomarker test membranes and/or biomarker testing devices. In some embodiments, the biomarker(s) assayed by additional biomarker test membrane(s) is determined based on initial test results.

Several embodiments relate to a kit comprising a case as described herein, one or more test membranes and instructional material. In some embodiments, the kit comprises a case, one or more test membranes, a NAF collection membrane and instructional material. In some embodiments, the kit comprises a case, one or more test membranes, a container of dilution buffer and instructional material. In some embodiments, the kit comprises a case, one or more test membranes, a collection membrane, a nipple cap and instructional material. In some embodiments, the kit comprises a case, one or more test membranes, a nipple cap and instructional material. In some embodiments, the kit comprises a case, one or more test membranes, a collection membrane, a nipple cap, a container of dilution buffer and instructional material. In some embodiments, the kit comprises a case, one or more test membranes, a nipple cap, a container of dilution buffer and instructional material. Kits according to any of the aforementioned embodiments may further comprise one or more of pipettes, gloves, a survey (either paper or online) of general health information and family history, and an addressed envelop for returning used devices. The aforementioned kits may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more test membranes as described herein. The 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more test membranes included in any of the aforementioned kits may be selected based on factors such as race, ethnicity, age, previous test results, medical history, family history, genetic profile or other criteria. In some of the aforementioned embodiments, the kits comprise a set of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more test membranes and the user selects 1, 2, 3, 4, 5, 6, 7, 8, 9, 10, or more test strips for use based on criteria such as race, ethnicity, age, geographic location, previous test results, medical history, family history, genetic profile or other criteria. In some of the aforementioned embodiments, the kits comprise a set of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more test membranes that are preselected for an individual subject based on criteria such as race, ethnicity, age, geographic location, previous test results, medical history, family history, genetic profile or other criteria. In some of the aforementioned embodiments, the kits comprise a set of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more test membranes that are preselected for distribution to a particular geographic region based on criteria such as the racial profile, ethnic profile, genetic profile, age distribution or other characteristic of the population in the geographic area. In some of the aforementioned embodiments, the kit further comprises instructions for ordering additional biomarker test membranes.

The instructional material may provide instructions for expressing NAF by breast massage, instructions for testing the collected NAF with the provided device, instructions for selecting test membranes for use and/or may include addresses to internet sites. While instructional materials typically comprise written or printed materials they are not limited to such. Any medium capable of storing such instructions and communicating them to an end user is contemplated.

Methods of Using a Biomarker-Testing Device and/or Kit as Described Herein

In some embodiments, a NAF sample may be expressed directly into a well or test membrane compartment of a biomarker-testing device as described herein. In some embodiments, a NAF sample may be expressed directly onto a wicking member. In some embodiments, a NAF sample may be transferred to a well or test membrane compartment of a biomarker-testing device by pipetting, pouring or rinsing. In some embodiments, the NAF sample may be adsorbed onto a collection membrane that is then placed into a well or test membrane compartment of the biomarker-testing device. In some embodiments, a collection membrane is removed from a nipple cap following NAF expression by manual massage and placed into a well of a biomarker-testing device. In other embodiments, buffer is added to the nipple cap following NAF expression by manual massage and subsequently transferred to a well or test membrane compartment of a biomarker-testing device by pipetting, pouring or wicking.

A NAF-containing collection membrane may be added to a well or test membrane compartment of a biomarker-testing device or a separate container containing an amount of buffer. In some embodiments the NAF-containing collection membrane is placed into a dry well or test membrane compartment of a biomarker-testing device and an amount of buffer is added to the collection membrane.

In several embodiments, NAF contained in the collection membrane is eluted in buffer for a period of 30 seconds, 1, 2, 3, 4, 5, 6, 7, 8, 9, 10, 11, 12, 13, 14, 15 or 30 minutes, one hour, 12 hours or 24 hours prior to biomarker testing. A sample of NAF-containing eluant may be transferred to a well of a biomarker testing device that is coupled to one or more test membranes or directly to a test membrane compartment. In some embodiments, a wicking member of a biomarker testing device is contacted with a sample of NAF-containing eluant.

In some embodiments, an indicator reagent may be added to a well, test membrane compartment, or wicking member of a biomarker-testing device.

In some embodiments, the test result may be “read” directly by the user. In some embodiments, the biomarker-testing device may be provided to a medical professional or laboratory or returned to the manufacturer to determine test results.

### EXAMPLE 1

In a study conducted by the Dr. Susan Love Research Foundation, 1002 Chinese women self-applied a BAND-AID®-like medical device, called a “nipple cap”, over their nipples. With the assistance of a nurse in a Chinese hospital, study participants then attempted to express NAF using massage. Any expressed fluid was absorbed onto a polymacron-based membrane incorporated in the nipple cap, and the entire device was removed from the woman's breast and stored frozen at −80 degrees until analysis. Of the 1002 women in the study, 255 women expressed NAF, and 300 NAF samples were collected (some women had bilateral NAF).

NAF samples were assayed for the presence of three protein biomarkers implicated in breast cancer risk and progression: epidermal growth factor (EGF), C-reactive protein (CRP), and basic fibroblast growth factor (bFGF).

The NAF-containing membranes were individually submerged in a buffered solution in a contact lens case and allowed to soak in the buffer for several minutes to elute the biomarkers. Test membranes designed to detect the presence of EGF, CRP, or bFGF were placed in the buffer, and the presence or absence of the biomarker was recorded. This process was performed on 300 samples over one day by members of the Dr. Susan Love Research Foundation.

One sample was positive for EGF, one sample was positive for bFGF, and one sample was positive for both bFGF and CRP. This study established proof of concept that samples of NAF collected by the study participants could be successfully analyzed for biomarkers of interest.

### EXAMPLE 2

The polymacron-based membranes used in the nipple cap for the collection of NAF in Example 1 exhibited poor aqueous absorption properties. Membranes composed of different hydrophilic, low protein-binding materials, including, for example, glass microfiber, polyvinylidene fluoride (PVDF), mixed cellulose esters, and polycarbonate, are used to determine of NAF collection efficiency and biomarker elution. A phosphate-buffered saline (PBS) solution comprising three biomarkers, EGF, CRP, and bFGF, is added to test membranes composed of hydrophilic, low protein-binding materials to simulate NAF collection. The fluid absorption properties of the membranes, such as the rate of adsorption and adsorption capacity, are evaluated. The membranes are optionally stored and then soaked in a volume of elution buffer for a period of time. The presence of the biomarkers in the elution buffer is detected via dot blot using appropriate detection antibodies to determine the elution efficiency of the membranes. Membranes exhibiting high absorbance and efficient biomarker elution are selected for further evaluation with NAF.

Either frozen NAF obtained from a NAF bank or fresh NAF obtained from 20 volunteers is added to membranes identified as having favorable absorbance and elution characteristics. Absorption of NAF is evaluated and elution efficiency is determined as described above. A satisfactory collection membrane having sufficient absorbance and elution efficiency is identified for use as a NAF-collection membrane.

### EXAMPLE 3

A panel of substances present at increased levels in the NAF of women who have ductal carcinoma in situ, genetic breast cancer risk factors, or who have had cancer in the contralateral breast was identified as candidate biomarkers of breast cancer risk. See Table 1.

Optimized conditions for detecting each of the candidate biomarkers listed in Table 1 are determined by adding a solution comprising the candidate biomarker at a concentration comparable to that found in NAF to a collection membrane identified according to Example 2 above. Collection membranes containing the candidate biomarkers are stored for a period of time, for example, 1 day, 1 week, 1 month, 6 months or 1 year, under a variety of conditions, for example, at 4° C., 0° C. or room temperature, or added directly to an amount of buffer sufficient to elute the candidate biomarker. Conditions for detection of the eluted candidate biomarkers are determined via dot blot assay using commercially available antibodies that bind to the biomarker or, for nucleotide biomarkers, complementary nucleotide probes.

Using the conditions determined above for candidate biomarker detection, one hundred NAF samples obtained from premenopausal healthy, high-risk, and breast-cancer-diagnosed subjects are tested for the presence of the candidate biomarker. Five to seven candidate biomarkers are selected on the basis of ease of detection, positive correlation with increased risk of having had developed or developing breast cancer, etc., for inclusion in one or more biomarker testing devices and further evaluation.

The ability of women to perform unsupervised at home biomarker screening on NAF is evaluated by providing premenopausal breast cancer survivors with a biomarker-testing device, a nipple cap comprising a well containing a collection membrane and instructions for expressing NAF by breast massage and for testing the collected NAF with the provided device. This population of subjects is selected in anticipation that their contralateral breast will be at increased risk for having had developed or developing breast cancer. The subjects are required to complete a survey (either paper or online) in which they provide general health information, family history, their test results, including a photograph of the device after they have performed the test, and feedback on the testing process. Used devices are collected to confirm test results. The subjects are evaluated by a physician at defined intervals, for example, monthly, every six months, yearly, every two years, etc., during a relevant time period, for example 5, 10, 15, 20, 25, or 30 years, for breast cancer. Correlations between the incidence of breast cancer and the detected biomarkers are determined.

A study to evaluate whether at home testing of NAF with a panel of candidate biomarkers can identify subjects with an increased risk of having developed or developing breast cancer is conducted with a sufficient number of premenopausal women of average risk to allow statistical analysis. Study participants collect and test NAF using a provided biomarker-testing device and report the results at defined intervals, such a monthly, every six months, yearly, every two years, etc. Study participants are additionally monitored for the development of breast cancer over a relevant time period, for example 5, 10, 15, 20, 25, or 30 years. A panel of biomarkers showing a statistically significant correlation with increased breast cancer risk is identified.

1-15. (canceled)

16. A biomarker-testing device for analyzing nipple aspirate fluid (NAF) for biomarkers indicative of breast cancer risk, the device comprising:
a case, comprising, a well for receiving a sample of NAF, and one or more compartments coupled to the NAF receiving well, wherein the one or more compartments are configured to receive one or more test membranes; and
at least one test membrane, wherein said test membrane is configured to perform at least one assay that is capable of detecting the presence of at least one biomarker, said at least one biomarker being associated with breast cancer risk; and
wherein said device indicates a positive response upon detecting the at least one biomarker.

17. The device of claim 16, wherein one or more test membranes is configured to perform an assay that is capable of detecting EGF, an assay that is capable of detecting CRP, and an assay that is capable of detecting bFGF.

18. The device of claim 16, wherein at least one test membrane is configured to perform an assay that is capable of detecting a biomarker is selected from the group consisting of: 15-F2t-isoprostane; 2,6-Cyclolycopene-1,5-diol; 8-Epimer of prostaglandin F₂ₐ (8-iso-PGF(2α)); 8-Isoprostane; Albumin; alpha1-Acid glycoprotein (AAG); alpha1-Antichymotrypsin; alpha1-Antichymotrypsin; alpha1-Antitrypsin; alpha1B-Glycoprotein; alpha2-Glycoprotein(Zn), alpha2-HS-glycoprotein; alpha-Casein; alpha-Hemoglobin; alpha-Lactalbumin; alpha-L-fucosidase; Aluminum; Aminopeptidase N; Antigen p97-melanotransferrin; Apolipoprotein A-I; Apolipoprotein D; Apolipoprotein E; Basic fibroblast growth factor (bFGF); beta-2-Microglobulin; beta-Casein; Beta-globin; Beta-hemoglobin; Butyrophilin; Calgizzarin (S100 A11; MLN70); Calgranulin B (S100 A9); Cancer-associated serine protease-protecting peptide (CRISPP peptide); Carcinoembryonic antigen (CEA); Cathepsin D; CD14; CD34 (prominin); CD36; Ceruloplasmin (ferroxidase); Cholesterol; Cholesterol epoxides; Clusterin; Coagulation factor II; Collagen α1; Complement C3; Complement C4; Complement C7; Complement factor B; Complement factor D; C-reactive protein (CRP); Cyclooxygenase-2 (COX2); Dehydroepiandrosterone sulphate (DHEAS); Enhancer protein; Ephrin; Epidermal growth factor (EGF); Epidermal growth factor receptor 2 (HER-2/neu); Erythropoietin; Estradiol (E2); Estrone (E1); Ferritin; Fibrinogen gamma-B chain; Gelsolin; GOS reactivity; Gross cystic disease fluid protein 15 (GCDFP-15); Group IIa secretory phospholipase A2 (sPLA2-IIa); Haptoglobin; Hemopexin; Human glandular kallikrein 2 (hK2); Human glandular kallikrein 10 (hK10); Human glandular kallikrein 2 (hK2); Human glandular kallikrein 6 (hK6); Ig alpha chain (heavy); Ig gamma chain (heavy); Ig J chain; Ig kappa chain (light); Ig lambda chain (light); Ig mu chain (heavy); Immunoglobin (Ig); Immunoglobulin A (IgA); Immunoglobulin G (IgG); Immunoglobulin M (IgM); Insulin-like growth factor binding protein-3 (IGFBP-3); Interleukin-1 (IL-1); Interleukin-25 (IL-25); Interleukin-6 (IL-6); Iroquois-class homeodomain protein; kappa-Casein; Keratin 1; Keratin 2a; Lactate dehydrogenase enzymes (LDH); Lactoferrin; Lactose; Lactotransferrin; Leucine-rich α-2-glycoprotein; Lipophilin B; Macrophage inhibitory cytokine 1 (MIC-1); Mitogen-activated protein kinase (MAPK); Nuclear factor kappa B (NFκB); Osteopontin; p16; P-cadherin; Plasminogen activator inhibitor (PAI-1); Polymeric-immunoglobulin receptor; Progesterone; Prolactin; Prolactin-induced protein (GCDFP-15); Prostasin (serine protease 8); Prostate specific antigen (human glandular kallikrein 3) (PSA hK3); Prostate-specific antigen (PSA); Protein carbonyls; pS2; Pyruvate kinase isozyme M2; Retinoic acid receptor responder; S100 A11 (calgizzarin;MLN70); Superoxide dismutase-1; Testosterone; Thomsen-Freidenreich antigen (TF); Thymosin beta-4; Tn antigen (Tn); Transcobalamin I; Transferrin; Transforming growth factor-alpha (TGFα); Tumor necrosis factor alpha (TNFα); Tumor necrosis factor receptor (TNFR); Tumor-associated antigen 90K (Mac2 BP); Urokinase-type plasminogen activator (uPA); Urokinase-type plasminogen activator receptor (uPAR); Vascular endothelial growth factor (VEGF); Vitamin D-binding protein precursor; and Zinc alpha2-glycoprotein.

19. The device of claim 16, wherein at least one assay employs a specific binding interaction.

20. The device of claim 19, wherein at least one test membrane comprises at least one capture reagent that specifically interacts with a complementary target biomarker, wherein at least one capture reagent is immobilized on at least one test membrane in at least one reaction zone, and an indicator reagent that is capable of specifically interacting with a biomarker/capture reagent complex to produce a detectable signal indicating the presence of a target biomarker in a NAF sample applied to the test membrane.

21. The device of claim 20, wherein the indicator reagent comprises a first antibody conjugated with gold to form a conjugate and the capture reagent comprises second antibody.

22. The device of claim 21, wherein application of a NAF sample to the test membrane at least partially dissolves the conjugate to form a liquid mixture, wherein at least a portion of the liquid mixture is adapted to move along the test membrane by capillary action and contact the second antibody in the reaction zone, wherein a sandwich of the conjugate and the second antibody will form if the targeted biomarker is present in the NAF sample, and wherein a detectable signal will develop upon concentration of the conjugate at the reaction zone.

23. The device of claim 16, wherein the positive response indicates an increased risk of breast cancer in a subject with a specificity and/or sensitivity of at least 70%, 75% 80%, 85%, 90%, 95%, 96%, 97%, 98% or 99% and a confidence interval of at least 90%, 95%, 96%, 97%, 98% or 99%.

24. The device of claim 16, wherein at least one test membrane is configured to perform 1, 2, 3, 4, 5, 6, 7, 8, 9, 10, 11, 12, 13, 14, 15, 16, 17, 18, 19, 20 or more biomarker detection assays.

25. The device of claim 16, wherein the device is configured for single use.

26. A kit comprising the device of claim 16 and corresponding instructions for use.

27. The kit of claim 26, further comprising a NAF collection membrane, a nipple cap, and elution buffer.

28. A method of identifying a biomarker of breast cancer risk having high sensitivity and specificity in identifying breast cancer risk, comprising:
a) obtaining NAF samples from women having a range of breast cancer risk levels;
b) testing the NAF samples for the presence or absence of a target biomarker;
c1) correlating the presence of the target biomarker in NAF with a particular risk level, or
c2) correlating the absence of the target biomarker in NAF with a particular risk level;
d1) determining whether the presence of the target biomarker in NAF is capable of identifying breast cancer risk with a specificity of at least 70%, 75% 80%, 85%, 90%, 95%, 96%, 97%, 98% or 99% and a confidence interval of at least 90%, 95%, 96%, 97%, 98% or 99%, or
d2) determining whether the absence of the target biomarker in NAF is capable of identifying breast cancer risk with a specificity of at least 70%, 75% 80%, 85%, 90%, 95%, 96%, 97%, 98% or 99% and a confidence interval of at least 90%, 95%, 96%, 97%, 98% or 99%; and
e) selecting the biomarker of breast cancer risk.

29. A method of identifying a biomarker panel having high sensitivity and specificity in identifying breast cancer risk, comprising:
a) obtaining NAF samples from women having a range of breast cancer risk levels;
b) testing the NAF samples for the presence or absence of 2, 3, 4, 5, 6, 7, 8, 9, 10, 11, 12, 13, 14, 15, 16, 17, 19, 20 or more target biomarkers;
c1) correlating the presence of a particular target biomarker with the presence or absence of one or more other target biomarkers in NAF obtained from women of a particular risk level, or
c2) correlating the absence of a particular target biomarker with the presence or absence of one or more other target biomarkers in NAF obtained from women of a particular risk level; and
d) selecting a combination of target biomarkers, wherein the combination is capable of identifying breast cancer risk with a specificity or sensitivity of at least 70%, 75% 80%, 85%, 90%, 95%, 96%, 97%, 98% or 99% and a confidence interval of at least 90%, 95%, 96%, 97%, 98% or 99%.

30. The method of claim 29, wherein at least one target biomarker is selected from the group consisting of: 15-F2t-isoprostane; 2,6-Cyclolycopene-1,5-diol; 8-Epimer of prostaglandin F₂ₐ (8-iso-PGF(2α)); 8-Isoprostane; Albumin; alpha1-Acid glycoprotein (AAG); alpha1-Antichymotrypsin; alpha1-Antichymotrypsin; alpha1-Antitrypsin; alpha1B-Glycoprotein; alpha2-Glycoprotein(Zn), alpha2-HS-glycoprotein; alpha-Casein; alpha-Hemoglobin; alpha-Lactalbumin; alpha-L-fucosidase; Aluminum; Aminopeptidase N; Antigen p97-melanotransferrin; Apolipoprotein A-I; Apolipoprotein D; Apolipoprotein E; Basic fibroblast growth factor (bFGF); beta-2-Microglobulin; beta-Casein; Beta-globin; Beta-hemoglobin; Butyrophilin; Calgizzarin (S100 A11; MLN70); Calgranulin B (S100 A9); Cancer-associated serine protease-protecting peptide (CRISPP peptide); Carcinoembryonic antigen (CEA); Cathepsin D; CD14; CD34 (prominin); CD36; Ceruloplasmin (ferroxidase); Cholesterol; Cholesterol epoxides; Clusterin; Coagulation factor II; Collagen α1; Complement C3; Complement C4; Complement C7; Complement factor B; Complement factor D; C-reactive protein (CRP); Cyclooxygenase-2 (COX2); Dehydroepiandrosterone sulphate (DHEAS); Enhancer protein; Ephrin; Epidermal growth factor (EGF); Epidermal growth factor receptor 2 (HER-2/neu); Erythropoietin; Estradiol (E2); Estrone (E1); Ferritin; Fibrinogen gamma-B chain; Gelsolin; GOS reactivity; Gross cystic disease fluid protein 15 (GCDFP-15); Group IIa secretory phospholipase A2 (sPLA2-IIa); Haptoglobin; Hemopexin; Human glandular kallikrein 2 (hK2); Human glandular kallikrein 10 (hK10); Human glandular kallikrein 2 (hK2); Human glandular kallikrein 6 (hK6); Ig alpha chain (heavy); Ig gamma chain (heavy); Ig J chain; Ig kappa chain (light); Ig lambda chain (light); Ig mu chain (heavy); Immunoglobin (Ig); Immunoglobulin A (IgA); Immunoglobulin G (IgG); Immunoglobulin M (IgM); Insulin-like growth factor binding protein-3 (IGFBP-3); Interleukin-1 (IL-1); Interleukin-25 (IL-25); Interleukin-6 (IL-6); Iroquois-class homeodomain protein; kappa-Casein; Keratin 1; Keratin 2a; Lactate dehydrogenase enzymes (LDH); Lactoferrin; Lactose; Lactotransferrin; Leucine-rich α-2-glycoprotein; Lipophilin B; Macrophage inhibitory cytokine 1 (MIC-1); Mitogen-activated protein kinase (MAPK); Nuclear factor kappa B (NFκB); Osteopontin; p16; P-cadherin; Plasminogen activator inhibitor (PAI-1); Polymeric-immunoglobulin receptor; Progesterone; Prolactin; Prolactin-induced protein (GCDFP-15); Prostasin (serine protease 8); Prostate specific antigen (human glandular kallikrein 3) (PSA hK3); Prostate-specific antigen (PSA); Protein carbonyls; pS2; Pyruvate kinase isozyme M2; Retinoic acid receptor responder; S100 A11 (calgizzarin;MLN70); Superoxide dismutase-1; Testosterone; Thomsen-Freidenreich antigen (TF); Thymosin beta-4; Tn antigen (Tn); Transcobalamin I; Transferrin; Transforming growth factor-alpha (TGFα); Tumor necrosis factor alpha (TNFα); Tumor necrosis factor receptor (TNFR); Tumor-associated antigen 90K (Mac2 BP); Urokinase-type plasminogen activator (uPA); Urokinase-type plasminogen activator receptor (uPAR); Vascular endothelial growth factor (VEGF); Vitamin D-binding protein precursor; and Zinc alpha2-glycoprotein.
